(19) United States
(12) Patent Application Publication
(10) Pub. No.: US 2011/0071364 A1
(43) Pub. Date: Mar. 24, 2011

(54) Remote Patient Monitoring System and Method Thereof

(75) Inventors: Bo-Jau KUO, Taipei (TW); Ching-Hsiu YANG, Taipei (TW)

(73) Assignee: NATIONAL YANG MING UNIVERSITY, Taipei (TW)

(21) Appl. No.: 12/624,275

(22) Filed: Nov. 23, 2009

(30) Foreign Application Priority Data
Sep. 18, 2009 (TW) 098131510

(51) Int. Cl. A61B 5/00

(52) U.S. Cl. 600/300

(57) ABSTRACT

This invention relates to a remote patient monitoring system and method thereof. The remote patient monitoring system comprises a portable electronic device, a remote monitor and a bio-signal detecting device which further comprises a plurality of bio-signal sensing modules, a wireless transmitting module and a control unit. The bio-signal detecting device is used for wearing on the body of user so that the bio-signal sensing modules can obtain a plurality of bio-signals form the user. The control unit is coupled to the bio-signal sensing modules and the wireless transmitting module. The control unit controls the wireless transmitting module to transmit the bio-signals to the portable electronic device. The portable electronic device transmits the bio-signals to the remote monitor.

### BACKGROUND OF THE INVENTION

1. Field of the Invention

This invention relates to a remote patient monitoring system, and more particularly, to a remote patient monitoring system and the method thereof.

2. Prior Art

In medical practice, many kinds of diseases require long-term observation and constant monitoring and alert so that the patient receives the best care. Take the monitoring of heart diseases, fever, and all kinds of pains for example, long-term observation is required on various physiological phenomena so that evaluation can be made on patients' condition and changes, and development of the disease can be controlled. However, long-term monitoring devices are not so patient-friendly which lead to low patient compliance. Furthermore, the insufficiency of devices and analyzing tools also limit the progress of medical practice.

Long-term bio-signal detecting system nowadays is built on the technique of traditional cable transmission. The tester must stick electrodes to the object, and the electrodes are connected by cables to the amplifier for analog-to-digital conversion, and then digital signal processing. It is very inconvenient for the tester due to the fact that the cables on the body, which limits the movement, even for going to the restroom. As a result, patients may have doubts for examination or not willing to conduct a long-term detection as doctor recommended.

In addition, most of the recording devices on the market are only suitable for hospital use instead of home use. Once being taken out of the hospital, the devices loss the function of prompt monitoring and then alert will not be sent out as well.

### SUMMARY OF THE INVENTION

The purpose of this invention is to provide a remote patient monitoring system and the method thereof to improve the existing techniques.

Based on one of the characteristics, this invention provides a remote patient monitoring system for taking care of users (patients). This remote patient monitoring system comprises a portable electronic device, a remote monitor, and a bio-signal detecting device. The bio-signal detecting device further comprises a plurality of bio-signal sensing modules, a wireless transmitting module, and a control unit. The bio-signal detecting device can be worn directly on the body of user so that the bio-signal sensing modules can obtain a plurality of bio-signals from the user. The control unit is coupled to the bio-signal sensing modules and the wireless transmitting module. The control unit aforementioned controls the wireless transmitting module to transmit the bio-signals to the portable electronic device, such as by Bluetooth transmission. The portable electronic device can transmit several bio-signals to the remote monitor via telecommunication network or Ethernet.

In accordance with another characteristic of the present invention, a remote patient monitoring method is provided to take care of users (patients). This method may be arranged with the bio-signal detecting device which can be worn on the user, the portable electronic device, and the remote monitor. This remote patient monitoring method comprises: using the bio-signal detecting device to obtain a plurality of bio-signals from the user, using the bio-signal detecting device to transmit the aforementioned bio-signals to the portable electronic device, and using the portable electronic device to transmit the aforementioned bio-signals to the remote monitor.

The advantage of the present invention is that the monitoring of the patient's condition and the medical evaluation can be in progress simultaneously at a remote end. Therefore, the disease progress can be thoroughly comprehended and the medication management system can conduct medication recommendation and evaluation based on the received bio-signals. In addition, personal medication alert and long-term bio-signal recording and monitoring system are also provided for the doctors for a more convenient medical evaluation, and many objective bio-signals also help the doctors to conduct a more precise evaluation. Furthermore, the automatic alert function of this invention can remind users and remote medical staff to pay attention, and conduct correct commands and responses.

The foregoing aspects and many of the attendant advantages of this invention will become more readily appreciated as the same becomes better understood by reference to the following detailed description, when taken in conjunction with the accompanying drawings.

### BRIEF DESCRIPTION OF THE DRAWINGS

FIG. 1 illustrates the remote patient monitoring system of an exemplary according to a preferred embodiment of the present invention;

FIG. 2 illustrates the functional block diagram of the bio-signal detecting device of an exemplary in a preferred embodiment of the present invention;

FIG. 3 illustrates the flowchart of the remote patient monitoring method of an exemplary in a preferred embodiment of the present invention; and

FIG. 4 illustrates the flowchart of the remote patient monitoring method of an exemplary in another preferred embodiment of the present invention.

### DETAILED DESCRIPTION OF THE PREFERRED EMBODIMENT

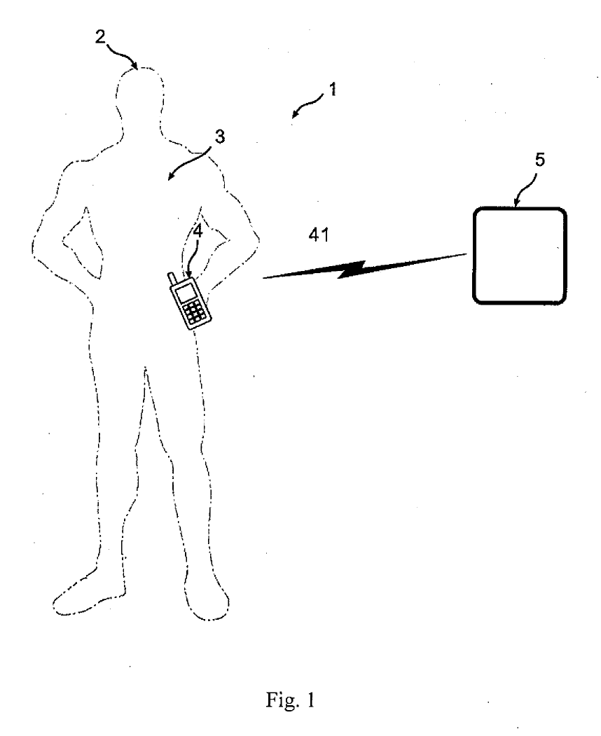

FIG. 1 is the remote patient monitoring system according to the preferred embodiment of the present invention. The remote patient monitoring system 1 in this embodiment comprises a bio-signal detecting device 3, a portable electronic device 4, and a remote monitor 5, wherein the portable electronic device 4 further comprises a output unit 41.

In this embodiment, the bio-signal detecting device 3 is used to be worn on the body of the user 2, such as between the chest and the abdomen. However, the wearing position of the bio-signal detecting device 3 is not limited in this invention. In the embodiment, the portable electronic device 4 can be a cell phone, a PDA, a notebook computer, or a netbook, and there is no limitation in this invention. In this embodiment, the output unit 41 can be the display monitor or the audio output device of the portable electronic device 4. In the preferred embodiment, the remote monitor 5 can be located in the hospital control room, or be the computer of family doctor or medical staff. This invention does not limit the type of the remote monitor 5.

In the preferred embodiment, the bio-signal detecting device 3 communicates with the portable electronic device 4 via wireless transmission. For example, the bio-signal detecting device 3 transmits the bio-signals or relevant information to the portable electronic device 4 via Bluetooth transmission. Nevertheless, this invention is not limited to the wireless transmission described above. In this embodiment, the portable electronic device 4 communicates with the remote monitor 5 via cable or wireless transmission. For example, the portable electronic device 4 can transmit information to the remote monitor 5 via telecommunication network or Ethernet.

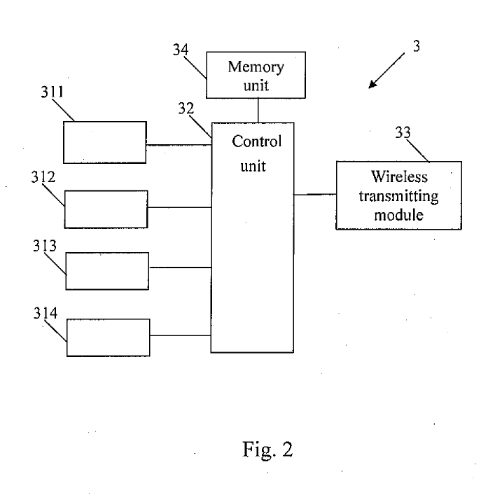

FIG. 2 is the functional block diagram of the bio-signal detecting device in a preferred embodiment of the present invention. Please also refer to FIG. 1 regarding the description of FIG. 2. The bio-signal detecting device 3 in this embodiment comprises a plurality of bio-signal detecting devices 311, 312, and 313, the acceleration sensor 314, the control unit 32, the wireless transmitting module 33, and the memory unit 34. The control unit 32 described above respectively couples to the bio-signal detecting devices 311, 312, and 313, the acceleration sensor 314, the wireless transmitting module 33, and the memory unit 34.

In the preferred embodiment, the bio-signal detecting devices 311, 312, and 313 obtain a plurality of bio-signals from user 2. Preferably, the bio-signal detecting devices 311, 312, and 313 conveniently receive the bio-signals by directly contacting the skin or the body of user 2, instead of receiving the bio-signals via external electrodes or cables. Nevertheless, it is not limited in this invention.

In the preferred embodiment, the bio-signal detecting devices 311, 312, and 313 can be a body temperature detecting device, an electrocardiogram detecting device, and an electromyogram detecting device for obtaining the body temperature signal, the electrocardiogram signal, and the electromyogram signal. However, in other embodiments, the bio-signal detecting device can also be other detecting devices such as respiratory detecting device. This invention does not prescribe limits on the type of the bio-signal detecting devices.

In the preferred embodiment, the acceleration sensor 314 can be a 3-axis accelerometer to provide the activity information of the user 2. For example, when the user 2 is walking, the detected value from the 3-axis of the acceleration sensor 314 changes; therefore, at least one detected value will be output to the control unit 32 for relevant signal processing so that we will know the user 2 is moving. On the other hand, if the user does not walk for a half day such as lying on the bed, the change of the 3-axis of the acceleration sensor 314 is minimum so that it is understood that the user 2 may suffer from an accident or illness and medical staff can provide assistance immediately

In this embodiment, the control unit 32 comprises the filter circuits, the amplifier circuits, the analog-to-digital conversion circuits, and the micro processors (not shown in figures). In this embodiment, the wireless transmitting module 33 can be a Bluetooth transmitting module, but there is no limitation in this invention.

In this preferred embodiment, the memory unit 34 can be non-volatile memory such as flash memory, but this invention does not restrict the type of the memory unit 34. In addition, the memory unit 34 in this embodiment can be used to store the physiological detected value and the activity information received from the bio-signal detecting devices 311, 312, and 313, and the acceleration sensor 314, wherein the physiological detected value is obtained from the bio-signals processed by the control unit 32, and activity information is obtained from the detected signals of acceleration sensor 314 processed by control unit 32. In this embodiment, the physiological detected value will also be referred as bio-signals for a clear description.

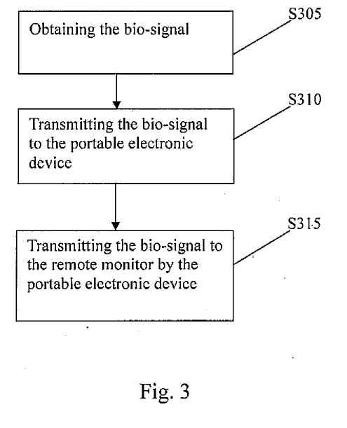

FIG. 3 is the flowchart of the remote patient monitoring method in a preferred embodiment of the present invention. The detailed description of FIG. 3 will conduct with FIG. 1 and FIG. 2.

In step S305, the bio-signal detecting device 3 worn on the body of the user 2 can obtain bio-signals by internally disposed bio-signal detecting devices 311, 312, and 313. In some embodiments, the bio-signal detecting device 3 can also use the internally disposed acceleration sensor 314 to obtain the detected value. In addition, the bio-signal detecting devices 311, 312, and 313, and the acceleration sensor 314 can transmit the detected signals to the control unit 32 for relevant signal processing in order to obtain the bio-signals and the activity information. The aforementioned bio-signals and the activity information can be stored in the memory unit 34.

In step S310, the control unit 32 controls the wireless transmitting module 33 for transmitting the bio-signals and/or the activity information to the portable electronic device 4.

In the preferred embodiment, the bio-signals and activity information is stored in the memory unit 34, and the control unit 32 can regularly, 5 minutes for example, transmit the bio-signals and/or the activity information stored in the memory unit 34 to the portable electronic device 4 via the wireless transmitting module 33. In other embodiments, the control unit 32 can also simultaneously transmit the bio-signals and/or the activity information to the portable electronic device 4 via the wireless transmitting module 33.

In step S315, the portable electronic device 4 transmits the bio-signals and/or the activity information to the remote monitor 5 via telecommunication network or Ethernet. Therefore, medical staff at remote end can keep an eye on the bio-signals of the user 2 by the remote monitor 5 for monitoring the condition, and immediate medical recommendation and evaluation can be promptly provided.

In the preferred embodiment, the portable electronic device 4 can be installed with application for immediate bio-signal analysis. Relevant medical recommendation can be provided via output unit 41 to remind the user 2. For example, if the body temperature of the user stays high, the portable electronic device 4 will remind the user 2 to take fever relieving pills every 4 hour or recommend the user 2 for hospitalization as soon as possible.

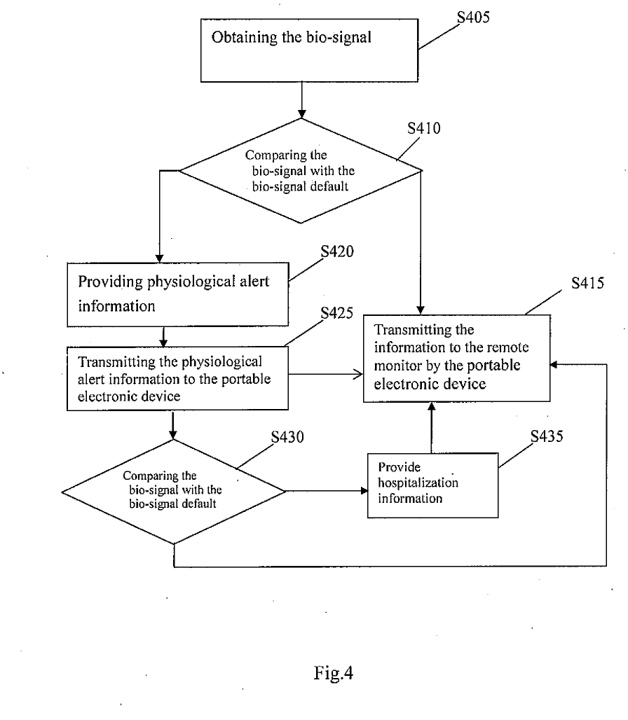

FIG. 4 is the flowchart of the remote patient monitoring method according to another embodiment of the present invention. The detailed description of FIG. 4 will conduct with FIG. 1 and FIG. 2.

In step S405, the bio-signal detecting device 3 worn on the body of user 2 can employ internally disposed bio-signal devices 311, 312, and 313 to obtain bio signals, wherein bio-signal devices 311, 312, and 313 transmit the detected signals to the control unit 32 for relevant signal processing to obtain bio-signals. The bio-signals aforementioned can be stored in the memory unit 34.

In step S410, the control unit 32 compares the bio-signals with the bio-signal default stored in the memory unit 34 so that physiological alert information can be provided for issuing warning or making judgment regarding the condition of the user 2.

For example, one of the received bio-signals by the control unit 32 is the body temperature signal and the body temperature is 37 degree Celsius. The control unit 32 compares the body temperature signal aforementioned with the bio-signal default, wherein the default can be a range, such as 37 to 37.5.

If the detected body temperature signal matches the bio-signal default stored in the memory unit 34, it indicates that the body temperature of the user 2 is normal and no medication is necessary. If each of the bio-signals matches the bio-signal default stored in the memory unit 34, the control unit 32 can control the wireless transmitting module 33 to directly transmit the bio-signals to the portable electronic device 4. The portable electronic device 4 then transmits the bio-signals to the remote monitor 5 to continuously record the condition of the user 2 (step S415).

If one of the detected bio-signals does not match the bio-signal default stored in the memory unit 34, such as a body temperature signal of 39.5 degree Celsius, the control unit will provide physiological alert information, wherein the physiological alert information can comprise a physiological alert message or a reminding message for medication (step S420).

In step S425, the control unit 32 controls the wireless transmitting module 33 to transmit the physiological alert information to the portable electronic device 4. The portable electronic device 4 transmits the physiological alert information to the remote monitor 5 to promptly warn the medical staff that the condition of the user 2 may worsen. In the preferred embodiment, when the portable electronic device 4 receives the physiological alert information, the portable electronic device 4 can remind the user 2 to take the medication, go to the hospital as soon as possible, or contact the family doctor via the output unit 41. In the preferred embodiment, when the remote monitor 5 receives the physiological alert information or the bio-signals, the remote monitor 5 can provide an analysis result to the portable electronic device 4 according to the physiological alert information or the bio-signals so that a simple medical suggestion can be provided to the user 2.

In step S430, the control unit 32 will continuously compare the bio-signals with the bio-signal default stored in the memory unit 34. If all the bio-signals match the bio-signal default, step S415 will proceed.

If the bio-signals aforementioned (body temperature signal) still do not match the bio-signal default, the control unit 32 will provide information of hospitalization. The control unit 32 will control the wireless transmitting module 33 to transmit the hospitalization information to the portable electronic device 4 (step S435). The portable electronic device 4 reminds the user 2 for hospitalization by the output unit 41. Moreover, the portable electronic device 4 will transmit the hospitalization information to the remote monitor 5 so that medical staff can provide on-site emergency care for the user 2 or pick up the user 2 to hospital for further medical care.

In another embodiment, the remote monitor 5 can also be stored up a plurality of bio-signal default. After the remote monitor 5 receives a plurality of bio-signals mentioned above, the remote monitor 5 will compare the aforementioned bio-signal default and bio-signals to provide the physiological alert information and/or the hospitalization information.

In conclusion, the remote patient monitoring system and the method thereof provided in the preferred embodiment of the present invention integrate the control unit, the bio-signal detecting module, and the wireless transmitting module with medication processing system so that objective and standardized physiological values are provided. As a result, medical staff can correctly evaluate the condition and the changes, and the patients' condition can be effectively controlled.

While the preferred embodiment of the invention has been illustrated and described, it will be appreciated that various changes can be made therein without departing from the spirit and scope of the invention.

What is claimed is:

1. A remote patient monitoring system for taking care of a user, the remote patient monitoring system comprising:
a portable electronic device;
a remote monitor; and
a bio-signal detecting device to be worn on the user, the bio-signal detecting device including:
a plurality of bio-signal sensing modules, to obtain a plurality of bio-signals from the user;
a wireless transmitting module; and
a control unit coupling to the bio-signal sensing modules and the wireless transmitting module respectively, the control unit controls the wireless transmitting module to transmit the bio-signals to the portable electronic device, and the portable electronic device transmits the bio-signals to the remote monitor.

2. The remote patient monitoring system as claimed in claim 1, wherein the portable electronic device is a cell phone, a PDA, or a notebook computer.

3. The remote patient monitoring system as claimed in claim 1, wherein the portable electronic device transmits the bio-signals to the remote monitor via a telecommunication network or Ethernet.

4. The remote patient monitoring system as claimed in claim 1, wherein the bio-signal detecting device further comprises a memory unit which couples to the control unit and is stored up a plurality of bio-signal default, the control unit compares the bio-signals with the bio-signal default to provide with the biological alert information, this control unit controls the wireless transmitting module to transmit the physiological alert information to the portable electronic device, the portable electronic device transmits the physiological alert information to the remote monitor.

5. The remote patient monitoring system as claimed in claim 4, wherein the physiological alert information comprises a physiological alert message or a reminding message for medication.

6. The remote patient monitoring system as claimed in claim 4, wherein the portable electronic device comprises an output unit which outputs the physiological alert message.

7. The remote patient monitoring system as claimed in claim 4, wherein the memory unit stores the bio-signals, the control unit simultaneously or periodically controls the wireless transmitting module to output the bio-signals to the portable electronic device.

8. The remote patient monitoring system as claimed in claim 4, wherein the remote monitor provides the result of the analysis based on the physiological alert information or the bio-signals to the portable electronic device.

9. The remote patient monitoring system as claimed in claim 4, wherein if one of the bio-signals does not match the value corresponding to the bio-signal default, then the control unit provides the physiological alert information.

10. The remote patient monitoring system as claimed in claim 9, wherein the control unit compares the bio-signals with the bio-signal default after a scheduled time, if one of the bio-signals still does not match the value corresponding to the bio-signal default, then the control unit provides a message for hospitalization, and the remote monitor receives the information of hospitalization.

11. The remote patient monitoring system as claimed in claim 1, wherein the remote monitor stores a plurality of bio-signal default, the remote monitor compares the bio-signal default with the bio-signals to provide with the physiological alert information.

12. The remote patient monitoring system as claimed in claim 1, wherein the bio-signals comprise a body temperature signal, an electrocardiogram signal, and an electromyogram signal.

13. The remote patient monitoring system as claimed in claim 1, wherein the bio-signal detecting device further comprises an acceleration sensor to provide activity information, the control unit controls the wireless transmitting module to transmit the activity information to the portable electronic device, the portable electronic device transmits the activity information to the remote monitor.

14. A remote patient monitoring method for taking care of a user, along with a bio-signal detecting device, a portable electronic device and a remote monitor, wherein the bio-signal detecting device is worn on the user, the remote patient monitoring method comprises:
using the bio-signal detecting device to obtain a plurality of bio-signals from the user;
using the bio-signal detecting device to transmit the bio-signals to the portable electronic device; and
using the portable electronic device to transmit the bio-signals to the remote monitor.

15. The remote patient monitoring method as claimed in claim 14, wherein the portable electronic device transmits the bio-signals to the remote monitor via a telecommunication network or Ethernet.

16. The remote patient monitoring method as claimed in claim 14 further comprises the comparison between the bio-signals and a plurality of bio-signal default for providing physiological alert information.

17. The remote patient monitoring method as claimed in claim 16, wherein the physiological alert information comprises a physiological warning message or a reminding message for medication.

18. The remote patient monitoring method as claimed in claim 16 further comprises: using the remote monitor to provide an analysis result to the portable electronic device according to the physiological alert information or the bio-signals.

19. The remote patient monitoring method as claimed in claim 16, wherein if one of the bio-signals does not match the value corresponding to the bio-signal default, the physiological alert information will be provided.

20. The remote patient monitoring method as claimed in claim 19, wherein the bio-signals are compared with the bio-signal default after a scheduled time, if one of the bio-signals still does not match the value corresponding to the bio-signal default, a message for hospitalization will be delivered.

21. The remote patient monitoring method as claimed in claim 14, wherein the bio-signals comprises a body temperature signal, an electrocardiogram signal, and an electromyogram signal.

22. The remote patient monitoring method as claimed in claim 14, wherein the bio-signal detecting device further comprises an acceleration sensor for providing activity information, the bio-signal detecting device transmits the activity information to the portable electronic device and the remote monitor.
